(19) United States
(12) Patent Application Publication
(10) Pub. No.: US 2023/0248339 A1
(43) Pub. Date: Aug. 10, 2023

(54) DEVICES AMD METHODS FOR SPINAL FACET JOINT FUSION

(71) Applicant: KIC VENTURES, LLC, MALDEN, MA (US)

(72) Inventors: KINGSLEY R. CHIN, MALDEN, MA (US); SHERIN KAMAL FETOUH, MALDEN, MA (US); JOSHUA FINKEL-LOPEZ, MALDEN, MA (US); OSCAR HERRERA, MALDEN, MA (US); RAHUL GAWANDE, MALDEN, MA (US); VITO LORE, MALDEN, MA (US)

(73) Assignee: KIC VENTURES, LLC, MALDEN, MA (US)

(21) Appl. No.: 18/107,474

(22) Filed: Feb. 8, 2023

Related U.S. Application Data
(60) Provisional application No. 63/308,140, filed on Feb. 9, 2022.

(51) Int. Cl. A61B 10/02; A61F 2/44

(52) U.S. Cl. CPC A61B 10/025; A61F 2/4455

(57) ABSTRACT

A surgical device includes an inner stylet component and an outer stylet component. The inner stylet component includes a first handle and an elongated rod having a proximal end attached to a bottom surface of the first handle and a distal end comprising a needle tip. The outer stylet component includes a second handle and an elongated cannula having a proximal end attached to a bottom surface of the second handle and a distal open end comprising cutting elements formed on an outer surface. The elongated rod is sized to removably fit and slide within the elongated cannula, and the first handle is configured to interlock with the second handle. Rotating the interlocked first and second handles moves forward the elongated cannula and the needle tip and cuts a profile in an insertion site with the cutting elements of the outer cannula.

### CROSS REFERENCE TO RELATED CO-PENDING APPLICATIONS

This application claims the benefit of U.S. Provisional Application Serial No. 63/308,140 filed February 9^{th}, 2022 and entitled “DEVICES AND METHODS FOR SPINAL FACET FUSION”, the contents of which are expressly incorporated herein by reference.

### FIELD OF THE INVENTION

The present invention relates to devices and methods for spinal facet joint fusion, and more particularly to devices used for accessing the intra-facet area of spinal facet joints and inserting bone graft material for fusing of the spinal facet joints.

### BACKGROUND OF THE INVENTION

The human spine includes individual vertebras that interlock with each other to form the spinal column. Under normal circumstances the spinal column functions to protect the neural structures and to allow us to stand erect, bear axial loads, and be flexible for bending and rotation. Inferior left and right facets of an upper vertebra fit perfectly into superior left and right facets of a lower vertebra below it, thereby forming left and right facet joints. The left and right facet joints provide stability and guide motion in the spine.

Pathologies of the facet joints are frequent causes of significant back and neck pains. A facet joint syndrome is an arthritic condition that is caused by degenerative changes to the facet joints. These degenerative changes cause the cartilage inside the facet joint to break down and become inflamed. Removal of the inflamed and degenerated cartilage and insertion of bone graft is used to treat the facet joint syndrome. The bone graft causes fusion of the diseased facet and relieves the pain.

Several spinal fixation systems exist for stabilizing the spine so that bony fusion is achieved. The majority of these fixation systems utilize fixation elements such as rods wires or plates that attach to screws threaded into the vertebral bodies, facets or the pedicles. In some fixation systems the facet joints are compressed together and attached together via spinal fixation elements 82a, 82b, shown in FIG. 1. However, in most prior art methods of facet fixation, compression and fixation does not result in actual bone-to-bone contact and fusion between superior and inferior facets. Accordingly, there is a need for a system and a method for bone-to-bone facet fixation that results in facet fusion between superior and inferior facets and spinal stabilization. It is also desirable to be able to implant the fusing implant percutaneously utilizing minimally invasive surgery.

### SUMMARY OF THE INVENTION

The present invention provides devices and methods used for accessing the intra-facet area of spinal facet joints and inserting bone graft material for fusing of the spinal facet joints.

In general, in one aspect, the invention features a surgical device including an inner stylet component and an outer stylet component. The inner stylet component includes a first handle and an elongated rod having a proximal end attached to a bottom surface of the first handle and a distal end comprising a needle tip. The outer stylet component includes a second handle and an elongated cannula having a proximal end attached to a bottom surface of the second handle and a distal open end comprising cutting elements formed on an outer surface. The elongated rod of the inner stylet component is sized to removably fit and slide within the elongated cannula of the outer stylet component, and the first handle is configured to sit onto and interlock with the second handle. The elongated rod is sized so that the needle tip protrudes through the distal open end of the elongated cannula, when the first and second handles are interlocked. Rotating the interlocked first and second handles moves forward the elongated cannula and the needle tip and cuts a profile in an insertion site with the cutting elements of the outer cannula and generates an opening in the insertion site with the needle tip.

Implementations of this aspect of the invention may include one or more of the following features. The needle tip comprises a trocar needle tip. The trocar needle tip comprises a three-sided cutting conical form. The trocar needle tip comprises one of a two-sided cutting blade, a bevel cutting blade, a flat form, a cone form, or a dual round cutting form. The cutting elements of the elongated cannula are configured to cut radially and/or straight into the insertion site. The cutting elements of the elongated cannula comprise quad-lead cutting threads and dual-lead cutting threads. The cutting elements of the elongated cannula comprise a ten-lead helix cutting reamer and a chamfer. The cutting elements of the elongated cannula comprise a ten-lead helix cutting burr and a chamfer. The cutting elements of the elongated cannula comprise a quad-lead helix cutting thread that has four sharp cutting edges. The cutting elements of the elongated cannula comprise four straight cutting flutes and a quad-lead helix cutting thread that has four sharp cutting edges. The distal open end of the elongated cannula further comprises one or more fenestrations configured to be used for delivering of graft material into the insertion site. The inner stylet component and the outer stylet component comprise one of titanium, cobalt, stainless steel, chrome, or alloys thereof, shape- memory alloy, or ceramic-metallic composite materials.

In general, in another aspect, the invention features a method for spinal facet joint fusion, including the following steps. First providing a surgical device that includes an inner stylet component and an outer stylet component. The inner stylet component includes a first handle and an elongated rod having a proximal end attached to a bottom surface of the first handle and a distal end comprising a needle tip. The outer stylet component includes a second handle and an elongated cannula having a proximal end attached to a bottom surface of the second handle and a distal open end comprising cutting elements formed on an outer surface. The elongated rod of the inner stylet component is sized to removably fit and slide within the elongated cannula of the outer stylet component, and the first handle is configured to sit onto and interlock with the second handle. The elongated rod is sized so that the needle tip protrudes through the distal open end of the elongated cannula, when the first and second handles are interlocked. Next, the method includes inserting the needle tip of the elongated rod into an intra-facet area of a facet joint, and then rotating the interlocked handles of the surgical device to distract the facet joint, and to generate an opening with the needle tip of the elongated rod, and to cut a radial profile with the cutting elements of the elongated cannula on the intra-facet area surfaces. Next, removing the elongated rod and inserting graft material into the opening via the elongated cannula. Finally, removing the elongated cannula from the facet joint leaving behind the graft material to cause fusion of the facet joint.

Implementations of this aspect of the invention may include one or more of the following features. The method may further include removing remnants of degenerated cartilage through the elongated cannula prior to inserting the graft material. The needle tip comprises a trocar needle tip. The trocar needle tip comprises a three-sided cutting conical form. The needle tip of the elongated rod is inserted into the intra-facet area of the facet joint percutaneously in a lateral to medial trajectory. The graft material comprises one of allograft bone material, synthetic bone growth promoting material, bone-polymer composite material, autograft bone material, xenograft bone material, polymers, “bio-glass” material, resorbable material, or non-resorbable material, or combinations thereof. The cutting elements of the elongated cannula are configured to cut radially and/or straight into the insertion site. The distal open end of the elongated cannula further comprises one or more fenestrations configured to be used for delivering the graft material into the insertion site.

The details of one or more embodiments of the invention are set forth in the accompanying drawings and description below. Other features, objects, and advantages of the invention will be apparent from the following description of the preferred embodiments, the drawings, and the claims.

### BRIEF DESCRIPTION OF THE DRAWINGS

Referring to the figures, wherein like numerals represent like parts throughout the several views:

FIG. 1 depicts a prior art system for facet fixation;

FIG. 1A is a perspective view of an embodiment of the spine fusion device according to this invention;

FIG. 1B is an enlarged view of the distal end of the spine fusion device of FIG. 1A;

FIG. 1C is bottom view of the distal end of the spine fusion device of FIG. 1A;

FIG. 1D is a perspective view of the stylet component of the spine fusion device of FIG. 1A;

FIG. 1E is an enlarged view of the distal end of the stylet rod of the spine fusion device of FIG. 1A;

FIG. 1F is a bottom view of the bone needle tip of the spine fusion device of FIG. 1A;

FIG. 2A is a perspective view of another embodiment of the spine fusion device according to this invention;

FIG. 2B is an enlarged view of the distal end of the spine fusion device of FIG. 2A;

FIG. 2C is bottom view of the distal end of the spine fusion device of FIG. 2A;

FIG. 3A is a perspective view of another embodiment of the spine fusion device according to this invention;

FIG. 3B is an enlarged view of the distal end of the spine fusion device of FIG. 3A;

FIG. 3C is bottom view of the distal end of the spine fusion device of FIG. 3A;

FIG. 4A is a perspective view of another embodiment of the spine fusion device according to this invention;

FIG. 4B is an enlarged view of the distal end of the spine fusion device of FIG. 4A;

FIG. 4C is bottom view of the distal end of the spine fusion device of FIG. 4A;

FIG. 5A is a perspective view of another embodiment of the spine fusion device according to this invention;

FIG. 5B is an enlarged view of the distal end of the spine fusion device of FIG. 5A;

FIG. 5C is bottom view of the distal end of the spine fusion device of FIG. 5A;

FIG. 6A is a perspective view of another embodiment of the spine fusion device according to this invention;

FIG. 6B is an enlarged view of the distal end of the spine fusion device of FIG. 6A;

FIG. 6C is bottom view of the distal end of the spine fusion device of FIG. 6A;

FIG. 7A is a perspective view of another embodiment of the spine fusion device according to this invention;

FIG. 7B is an enlarged view of the distal end of the spine fusion device of FIG. 7A;

FIG. 7C is bottom view of the distal end of the spine fusion device of FIG. 7A;

FIG. 8A is a perspective view of another embodiment of the spine fusion device according to this invention;

FIG. 8B is an enlarged view of the distal end of the spine fusion device of FIG. 8A;

FIG. 8C is bottom view of the distal end of the spine fusion device of FIG. 8A;

FIG. 9A is a perspective view of another embodiment of the stylet component of the spine fusion device of FIG. 1A;

FIG. 9B is an enlarged view of the distal end of the stylet rod of the stylet component of FIG. 9A;

FIG. 9C is a bottom view of the bone needle tip of the stylet rod of FIG. 9B;

FIG. 10A is a perspective view of another embodiment of the stylet component of the spine fusion device of FIG. 1A;

FIG. 10B is an enlarged view of the distal end of the stylet rod of the stylet component of FIG. 10A;

FIG. 10C is a bottom view of the bone needle tip of the stylet rod of FIG. 10B;

FIG. 11A is a perspective view of another embodiment of the stylet component of the spine fusion device of FIG. 1A;

FIG. 11B is an enlarged view of the distal end of the stylet rod of the stylet component of FIG. 11A;

FIG. 11C is a bottom view of the bone needle tip of the stylet rod of FIG. 11B;

FIG. 12A is a perspective view of another embodiment of the stylet component of the spine fusion device of FIG. 1A;

FIG. 12B is an enlarged view of the distal end of the stylet rod of the stylet component of FIG. 12A;

FIG. 12C is a bottom view of the bone needle tip of the stylet rod of FIG. 12B;

FIG. 13A is a perspective view of another embodiment of the stylet component of the spine fusion device of FIG. 1A;

FIG. 13B is an enlarged view of the distal end of the stylet rod of the stylet component of FIG. 13A;

FIG. 13C is a bottom view of the bone needle tip of the stylet rod of FIG. 13B;

FIG. 14 is a perspective view of the first method step of inserting the spine fusion device of FIG. 1A into the facet joint;

FIG. 15 is a perspective view of the second method step of rotating the handle of the spine fusion device of FIG. 1A;

FIG. 16 is a perspective view of the third method step of removing the stylet component of the spine fusion device of FIG. 1A;

FIG. 17 is a perspective view of the fourth method step of inserting the bone graft material into the facet joint through the cannula of the spine fusion device of FIG. 1A;

FIG. 18 is a perspective view of the fourth method step depicting the insertion of the bone graft material into the facet joint;

FIG. 19 is a perspective view of the fifth method step of removing the cannula component of the spine fusion device of FIG. 1A from the facet joint; and

FIG. 20 is a block diagram of the method of spinal fusion using the device od FIG. 1A, according to this invention.

### DETAILED DESCRIPTION OF THE INVENTION

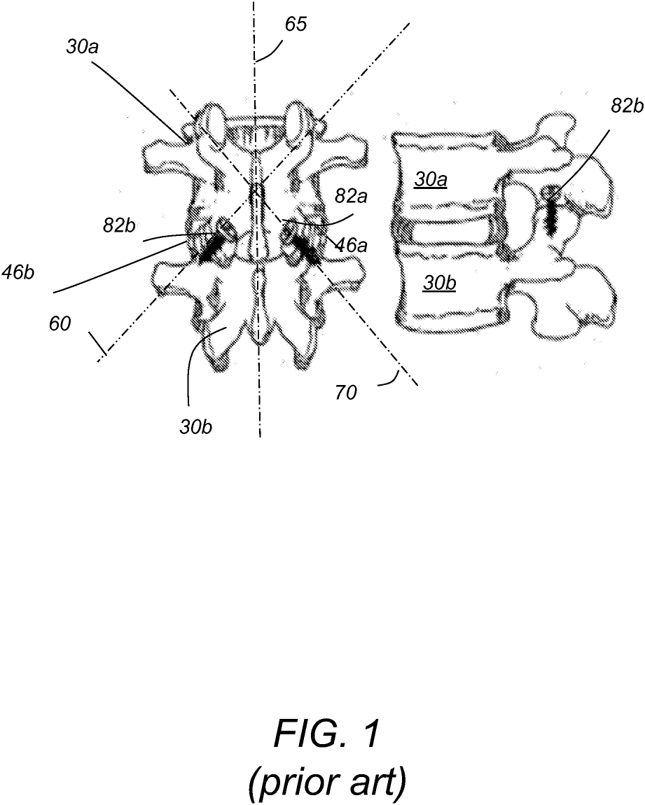

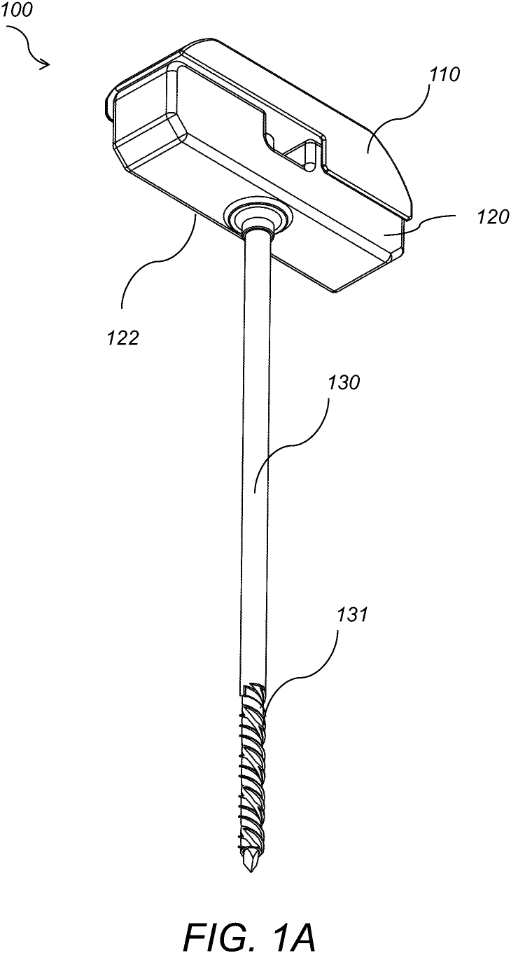

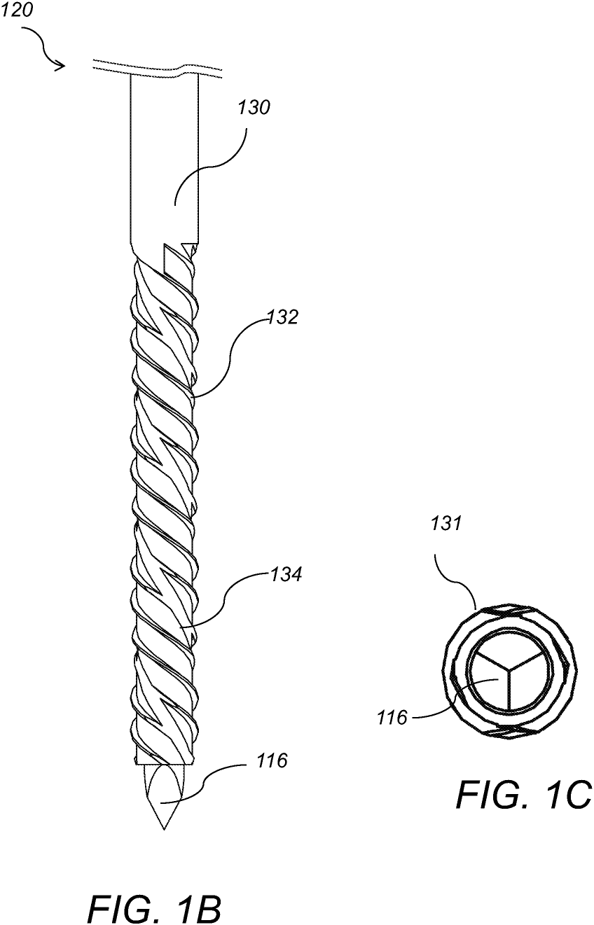

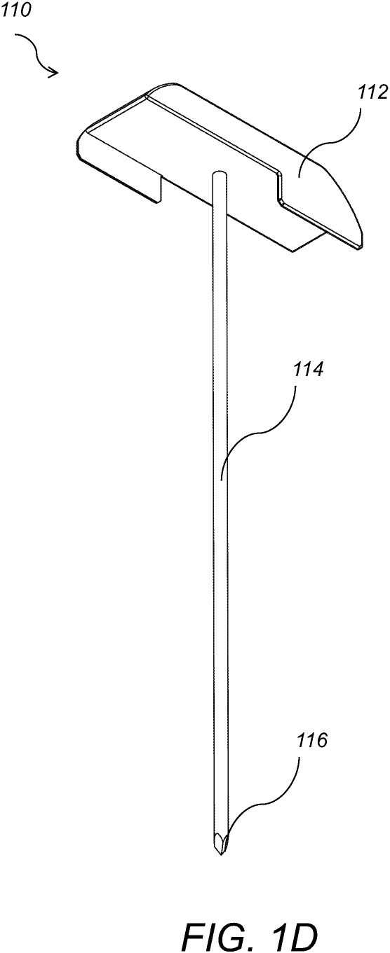

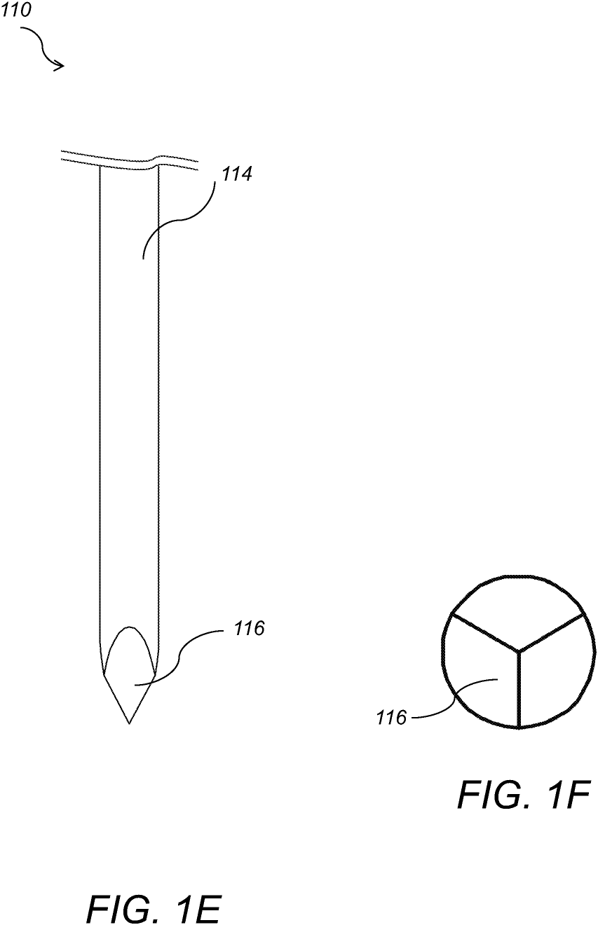

Referring to FIG. 1, spinal fixation elements 82a, 82b are used to secure together first and second facet joints 46a, 46b of two adjacent vertebras 30a and 30b. The spinal fixation elements 82a, 82b are inserted along directions 60, and to insert 70, respectively. In most cases, directions 60, 70 are symmetrically positioned to the left and right of the spinal midline 65. In this prior art example, fixation elements 82a, 82b are facet screws and are placed in a trans-facet way for connecting adjacent vertebras 30a, 30b. In other examples, fixation elements 82a, 82b, may be staples, wires, or pins, and they may connect adjacent or non-adjacent vertebras via trans-facet, intra-facet, trans-laminar, trans-facet-pedicular, trans-pedicular, or through any other vertebral location.

The present invention describes a new device used to provide access to the facet joints percutaneously in a lateral to medial trajectory and to insert bone graft material into the intra-facet area for fusing of the spinal facet joints. The bone graft material is made of allograft material, which may be actual bone material harvested from human donors, or synthetic material, or combination thereof.

Referring to FIG. 1A-FIG. 1E, device 100 includes an inner stylet component 110 and an outer component 120. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. The term trocar refers to a surgical instrument with a cutting needle tip. In this embodiment trocar needle tip 116 has a three-sided cutting conical form, as shown in FIG. 1F. Outer component 120 includes a handle 122 and an elongated cylindrical cannula 130. Elongated cannula 130 has a proximal end attached to a bottom surface of the handle 122 and a distal end 131 having cutting threads formed on its outer surface. The cutting threads may be single-lead threads, double-lead threads, triple-lead threads, quadruple-lead threads, or combinations thereof Single-lead threads are formed by cutting one groove with a single-point tool. A double-lead thread has two grooves, a triple-lead thread has three grooves, and a quadruple-lead thread has four grooves. The lead is the distance that the cutting thread travels in one revolution. In one turn a single-lead cutting thread moves forward the distance (pitch) of one thread. Similarly, in one turn a double-lead cutting thread, moves by two threads, a triple-lead thread moves by three threads, and a quadruple-lead thread moves by four threads. In this embodiment, distal end 131 includes a combination of quad-lead cutting threads 132, and dual-lead cutting threads 134, as shown in FIG. 1B. The elongated rod 114 of the inner stylet component 110 is sized to fit and slide within the elongated cannula 130 of the outer component 120, so that the distal trocar needle tip 116 protrudes through the distal open end 131 of the elongated cannula 130. The handle 112 of the inner stylet component 110 sits onto and interlocks with the handle 122 of the outer component 120. The two interlocked handles 112, 122 are used to rotate and move forward together the assembled cannula 130 with the trocar needle tip 116 of the inner stylet rod 114. The rotation of the interlocked handles 112, 122 causes the outer cutting threads 132, 134 of the cannula 130 to create a thread profile in the facet joints and moves the trocar needle tip 116 of the inner stylet rod 114 forward to create an opening. After the opening and the thread profile are created in the facet joints, the inner stylet rod is removed and the bone graft material is inserted through the cannula into the opening. In one example, the cannula 130 and the inner stylet rod have a length in the range of 50 mm to 150 mm and a diameter in the range of 3 mm to 12 mm.

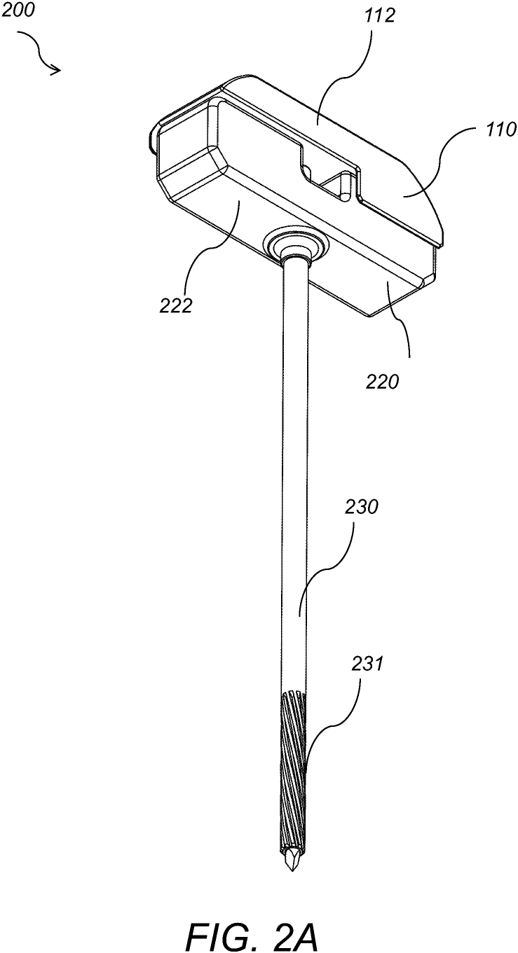

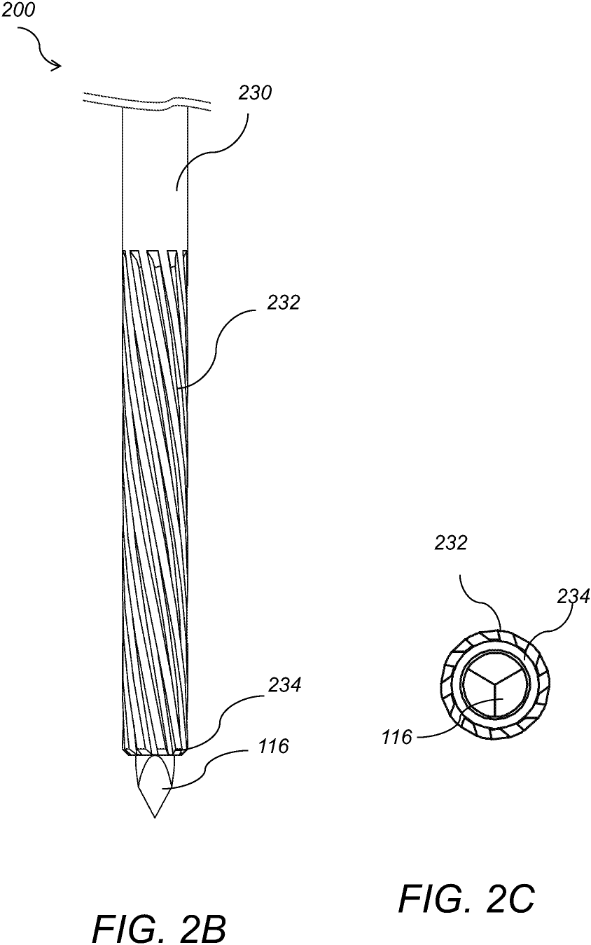

Referring to FIG. 2A-FIG. 2C, device 200 includes an inner stylet component 110 and an outer component 220. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. In this embodiment trocar needle tip 116 has a three-sided cutting conical form, as shown in FIG. 1F. Outer component 220 includes a handle 222 and an elongated cylindrical cannula 230. Elongated cannula 230 has a proximal end attached to a bottom surface of the handle 222 and a distal end 231 having a cutting surface formed on its outer surface. In this embodiment, distal end 231 includes a ten-lead helix cutting reamer 232, and a chamfer 234, as shown in FIG. 2B. The elongated rod 114 of the inner stylet component 110 is sized to fit and slide within the elongated cannula 230 of the outer component 220, so that the distal trocar needle tip 116 protrudes through the distal open end 231 of the elongated cannula 230. The handle 112 of the inner stylet component 110 sits onto and interlocks with the handle 222 of the outer component 220. The two interlocked handles 112, 222 are used to rotate and move forward together the assembled cannula 230 with the trocar needle tip 116 of the inner stylet rod 114. The rotation of the interlocked handles 112, 222 causes the reamer 232 of the cannula 230 to cut radially the material in the facet joints and moves the trocar needle tip 116 of the inner stylet rod 114 forward to create an opening. After the opening and the reamer profile are created in the facet joints, the inner stylet rod is removed and the bone graft material is inserted through the cannula into the opening.

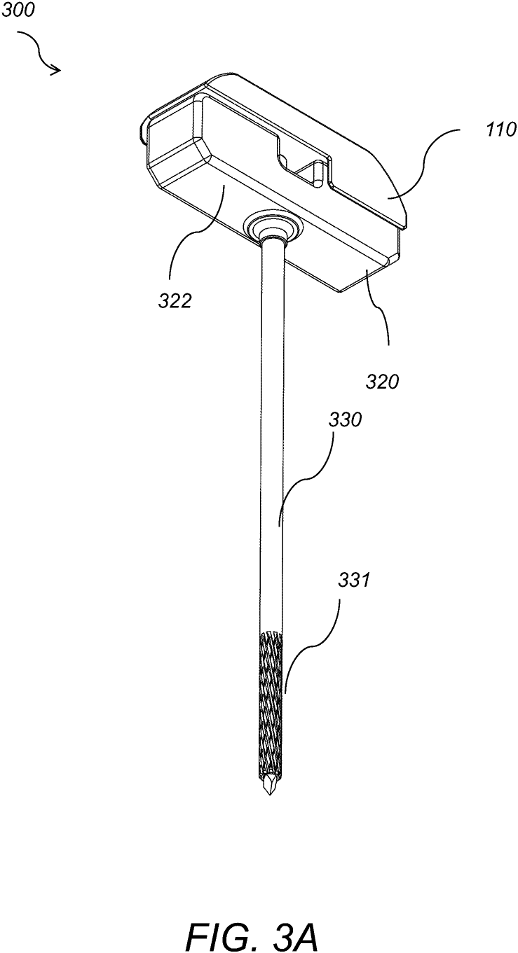

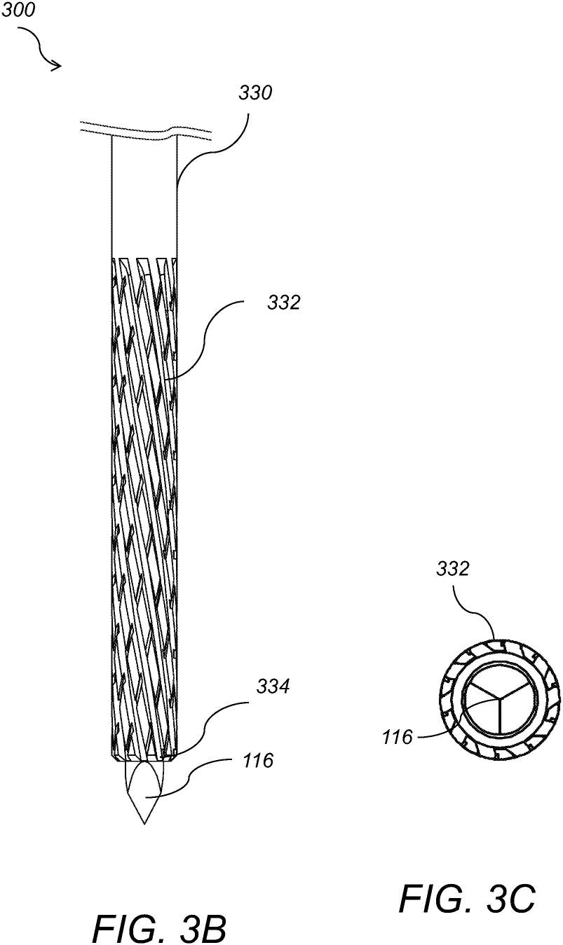

Referring to FIG. 3A-FIG. 3C, device 300 includes an inner stylet component 110 and an outer component 320. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. In this embodiment trocar needle tip 116 has a three-sided cutting conical form, as shown in FIG. 1F. Outer component 320 includes a handle 322 and an elongated cylindrical cannula 330. Elongated cannula 330 has a proximal end attached to a bottom surface of the handle 322 and a distal end 331 having a cutting surface formed on its outer surface. In this embodiment, distal end 331 includes a ten-lead helix cutting burr 332, and a chamfer 334, as shown in FIG. 3B. The elongated rod 114 of the inner stylet component 110 is sized to fit and slide within the elongated cannula 330 of the outer component 320, so that the distal trocar needle tip 116 protrudes through the distal open end 331 of the elongated cannula 330. The handle 112 of the inner stylet component 110 sits onto and interlocks with the handle 322 of the outer component 320. The two interlocked handles 112, 322 are used to rotate and move forward together the assembled cannula 330 with the trocar needle tip 116 of the inner stylet rod 114. The rotation of the interlocked handles 112, 322 causes the burr 332 of the cannula 330 to cut radially the material in the facet joints and moves the trocar needle tip 116 of the inner stylet rod 114 forward to create an opening. After the opening and the burr profile are created in the facet joints, the inner stylet rod is removed and the bone graft material is inserted through the cannula into the opening.

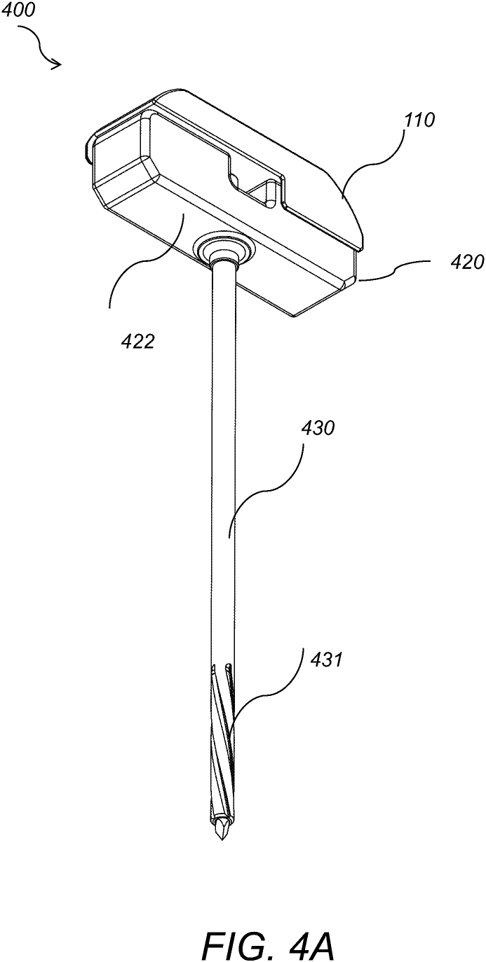

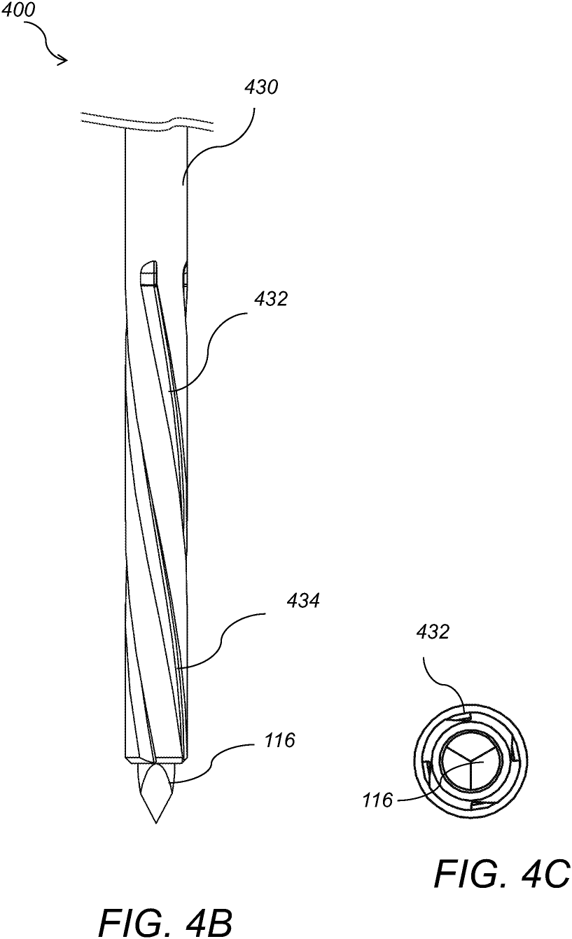

Referring to FIG. 4A-FIG. 4C, device 400 includes an inner stylet component 110 and an outer component 420. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. In this embodiment trocar needle tip 116 has a three-sided cutting conical form, as shown in FIG. 1F. Outer component 420 includes a handle 422 and an elongated cylindrical cannula 430. Elongated cannula 430 has a proximal end attached to a bottom surface of the handle 422 and a distal end 431 having a cutting surface formed on its outer surface. In this embodiment, distal end 431 includes a quad-lead helix cutting thread 432 that has four sharp cutting edges 434, as shown in FIG. 4B. The elongated rod 114 of the inner stylet component 110 is sized to fit and slide within the elongated cannula 430 of the outer component 420, so that the distal trocar needle tip 116 protrudes through the distal open end 431 of the elongated cannula 430. The handle 112 of the inner stylet component 110 sits onto and interlocks with the handle 422 of the outer component 420. The two interlocked handles 112, 422 are used to rotate and move forward together the assembled cannula 430 with the trocar needle tip 116 of the inner stylet rod 114. The rotation of the interlocked handles 112, 422 causes the burr 432 of the cannula 430 to cut radially the material in the facet joints and moves the trocar needle tip 116 of the inner stylet rod 114 forward to create an opening. After the opening and the thread profile are created in the facet joints, the inner stylet rod is removed and the bone graft material is inserted through the cannula into the opening.

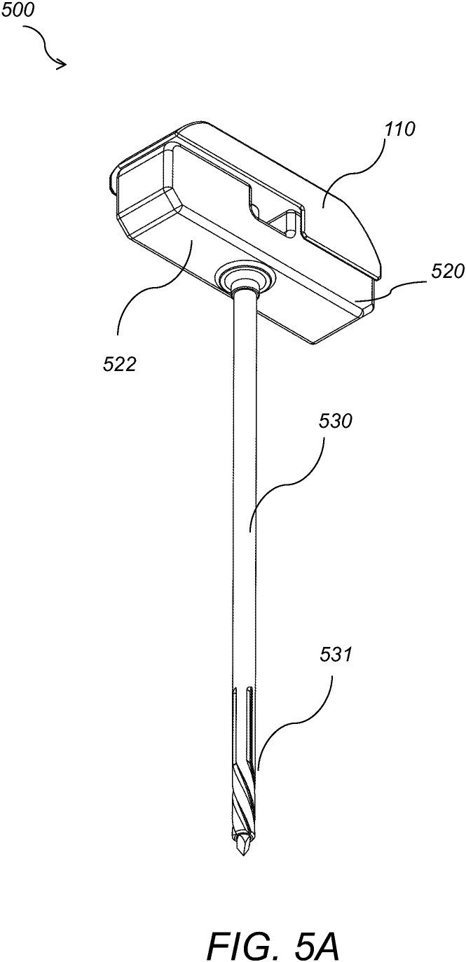

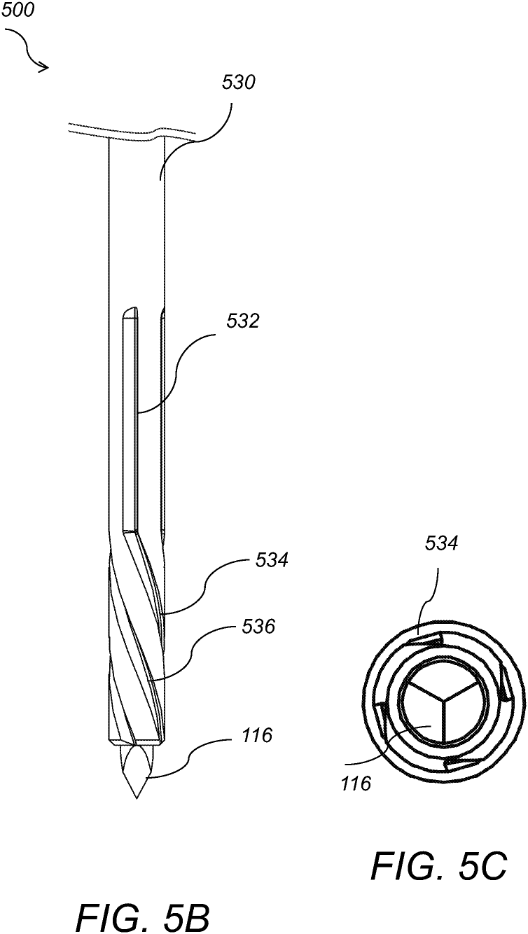

Referring to FIG. 5A-FIG. 5C, device 500 includes an inner stylet component 110 and an outer component 520. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. In this embodiment trocar needle tip 116 has a three-sided cutting conical form, as shown in FIG. 1F. Outer component 520 includes a handle 522 and an elongated cylindrical cannula 530. Elongated cannula 530 has a proximal end attached to a bottom surface of the handle 522 and a distal end 531 having a cutting surface formed on its outer surface. In this embodiment, distal end 531 includes combination of four straight cutting flutes 532 and a quad-lead helix cutting thread 536 that has four sharp cutting edges 534, as shown in FIG. 5B. The elongated rod 114 of the inner stylet component 110 is sized to fit and slide within the elongated cannula 530 of the outer component 520, so that the distal trocar needle tip 116 protrudes through the distal open end 531 of the elongated cannula 530. The handle 112 of the inner stylet component 110 sits onto and interlocks with the handle 522 of the outer component 520. The two interlocked handles 112, 522 are used to rotate and move forward together the assembled cannula 530 with the trocar needle tip 116 of the inner stylet rod 114. The rotation of the interlocked handles 112, 522 causes the cutting surface 531 of the cannula 530 to cut first radially and then straight the material in the facet joints and moves the trocar needle tip 116 of the inner stylet rod 114 forward to create an opening. After the opening and the thread profile are created in the facet joints, the inner stylet rod is removed and the bone graft material is inserted through the cannula into the opening.

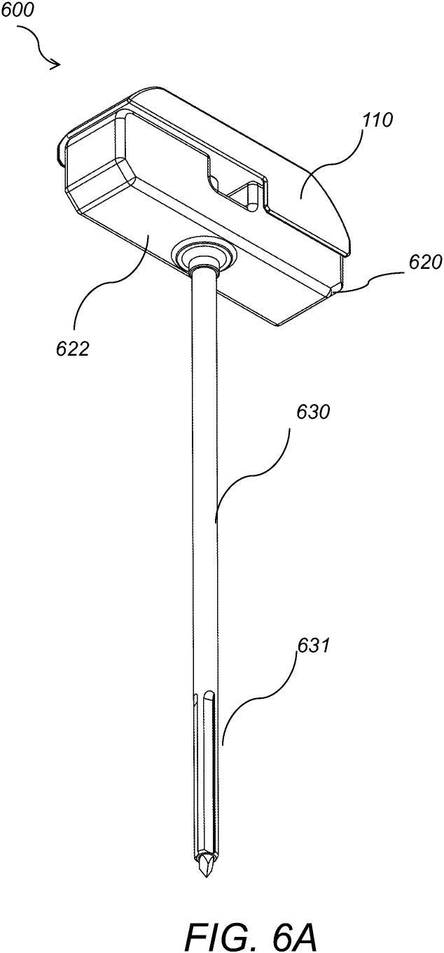

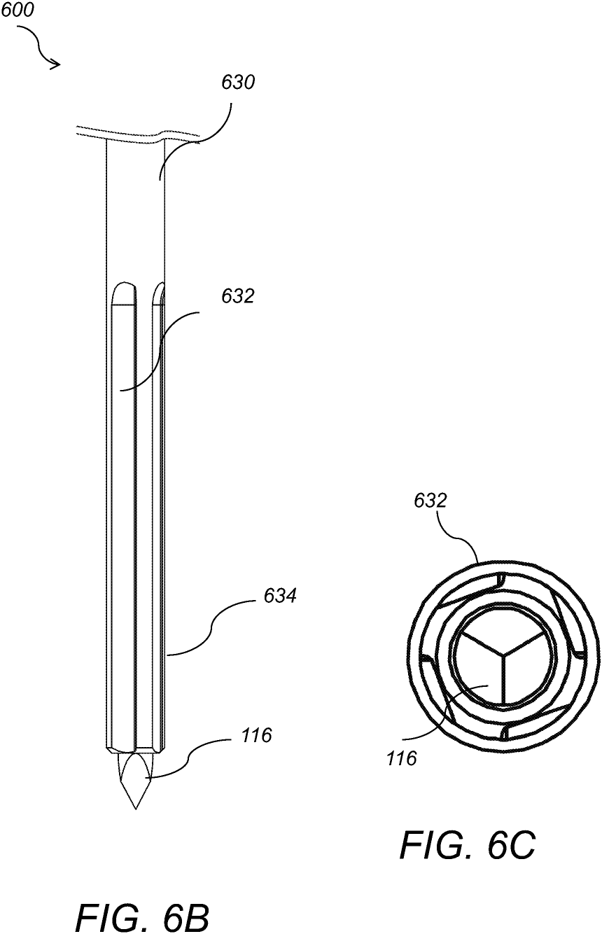

Referring to FIG. 6A-FIG. 6C, device 600 includes an inner stylet component 110 and an outer component 620. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. In this embodiment trocar needle tip 116 has a three-sided cutting conical form, as shown in FIG. 1F. Outer component 620 includes a handle 622 and an elongated cylindrical cannula 630. Elongated cannula 630 has a proximal end attached to a bottom surface of the handle 622 and a distal end 631 having a cutting surface 631a formed on its outer surface. In this embodiment, cutting surface 631a includes four straight cutting flutes 632 that have four sharp cutting edges 634, as shown in FIG. 6B. The elongated rod 114 of the inner stylet component 110 is sized to fit and slide within the elongated cannula 630 of the outer component 620, so that the distal trocar needle tip 116 protrudes through the distal open end 631 of the elongated cannula 630. The handle 112 of the inner stylet component 110 sits onto and interlocks with the handle 622 of the outer component 620. The two interlocked handles 112, 622 are used to rotate and move forward together the assembled cannula 630 with the trocar needle tip 116 of the inner stylet rod 114. The rotation of the interlocked handles 112, 622 causes the cutting surface 631a of the cannula 630 to cut straight the material in the facet joints and moves the trocar needle tip 116 of the inner stylet rod 114 forward to create an opening. After the opening and the cut profile are created in the facet joints, the inner stylet rod is removed and the bone graft material is inserted through the cannula into the opening.

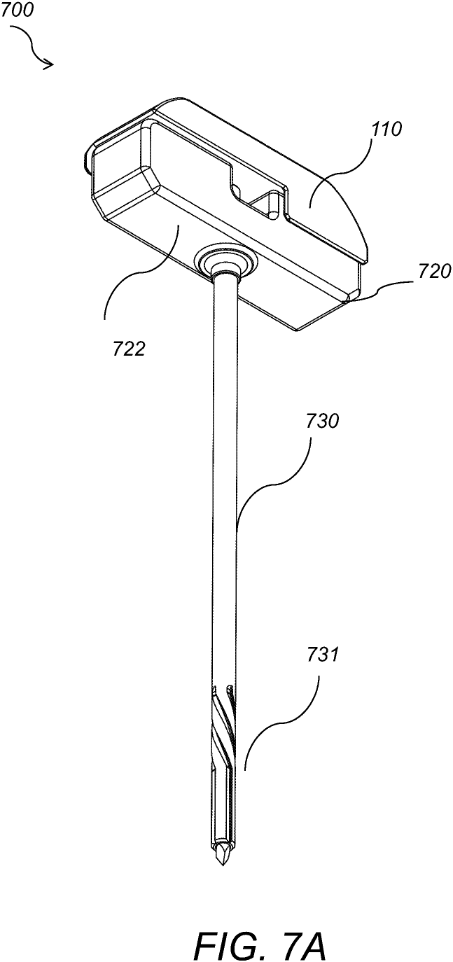

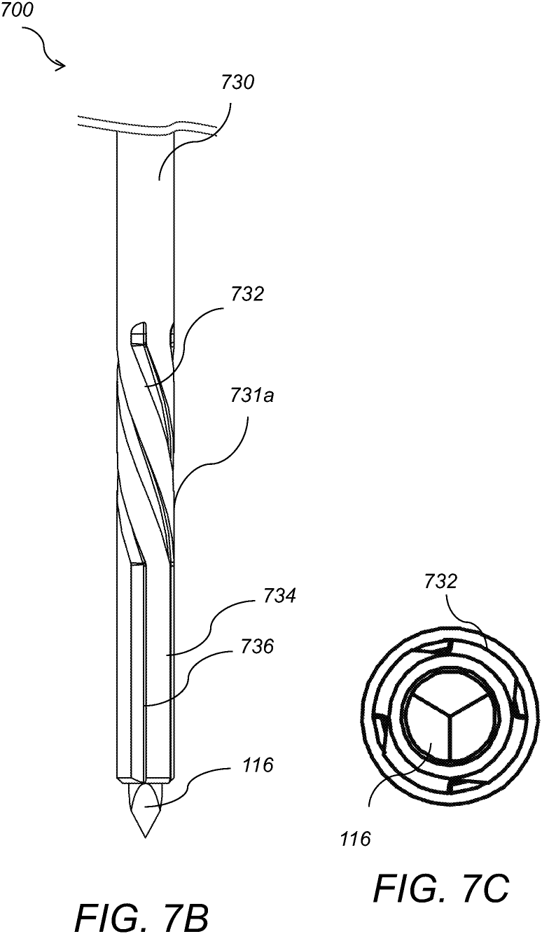

Referring to FIG. 7A-FIG. 7C, device 700 includes an inner stylet component 110 and an outer component 720. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. In this embodiment trocar needle tip 116 has a three-sided cutting conical form, as shown in FIG. 1F. Outer component 720 includes a handle 722 and an elongated cylindrical cannula 730. Elongated cannula 730 has a proximal end attached to a bottom surface of the handle 722 and a distal end 731 having a cutting surface 731a formed on its outer surface. In this embodiment, cutting surface 731a includes combination of four straight cutting flutes 736 and a quad-lead helix cutting thread 732 that has four sharp cutting edges 734, as shown in FIG. 7B. The elongated rod 114 of the inner stylet component 110 is sized to fit and slide within the elongated cannula 730 of the outer component 720, so that the distal trocar needle tip 116 protrudes through the distal open end 731 of the elongated cannula 730. The handle 112 of the inner stylet component 110 sits onto and interlocks with the handle 722 of the outer component 720. The two interlocked handles 112, 722 are used to rotate and move forward together the assembled cannula 730 with the trocar needle tip 116 of the inner stylet rod 114. The rotation of the interlocked handles 112, 722 causes the cutting surface 731a of the cannula 730 to cut first straight and then helically the material in the facet joints and moves the trocar needle tip 116 of the inner stylet rod 114 forward to create an opening. After the opening and the cut profile are created in the facet joints, the inner stylet rod is removed and the bone graft material is inserted through the cannula into the opening.

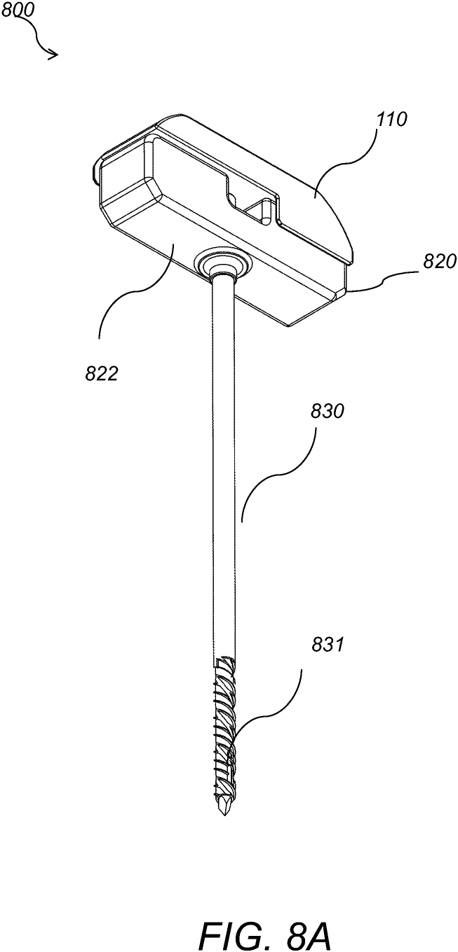

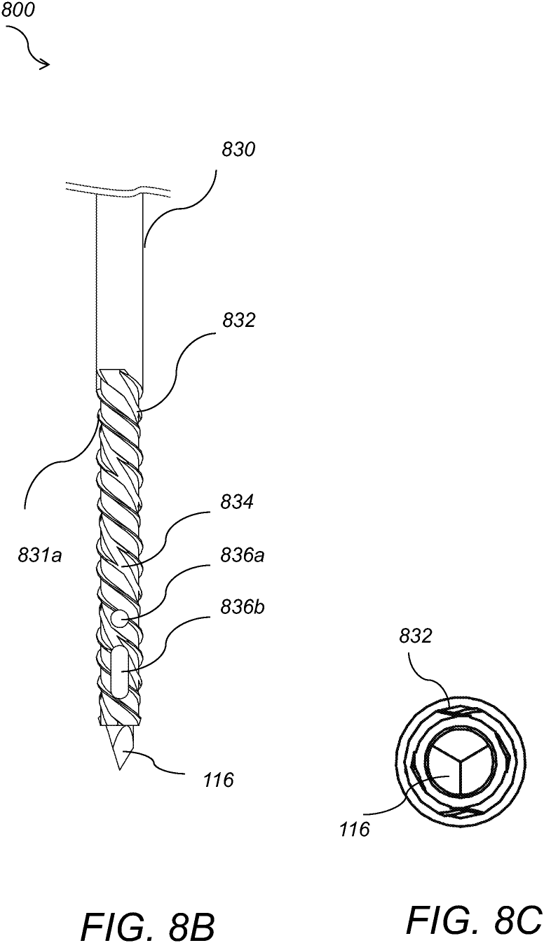

Referring to FIG. 8A-FIG. 8C, device 800 includes an inner stylet component 110 and an outer component 820. Inner stylet component 110 includes a handle 112 and an elongated rod 114. Elongated rod 114 has a proximal end attached to a bottom surface of the handle 112 and a distal end forming a trocar needle tip 116, as shown in FIG. 1D. In this embodiment trocar needle tip 116 has a three-sided cutting conical form, as shown in FIG. 1F. Outer component 820 includes a handle 822 and an elongated cylindrical cannula 830. Elongated cannula 830 has a proximal end attached to a bottom surface of the handle 822 and a distal end 831 having a cutting surface 831a formed on its outer surface. In this embodiment, cutting surface 831a includes a combination of quad-lead threads 832 and a dual-lead cutting threads 834, as shown in FIG. 7B. In this embodiment distal end 831 also includes circular 836a and oval 836b fenestrations that are used for the delivery of the graft material. The elongated rod 114 of the inner stylet component 110 is sized to fit and slide within the elongated cannula 730 of the outer component 820, so that the distal trocar needle tip 116 protrudes through the distal open end 831 of the elongated cannula 830. The handle 112 of the inner stylet component 110 sits onto and interlocks with the handle 822 of the outer component 820. The two interlocked handles 112, 822 are used to rotate and move forward together the assembled cannula 830 with the trocar needle tip 116 of the inner stylet rod 114. The rotation of the interlocked handles 112, 822 causes the cutting surface 831a of the cannula 830 to cut and create the thread profile in the material of the facet joints and moves the trocar needle tip 116 of the inner stylet rod 114 forward to create an opening. After the opening and the thread profile are created in the facet joints, the inner stylet rod is removed and the bone graft material is inserted through the cannula into the opening.

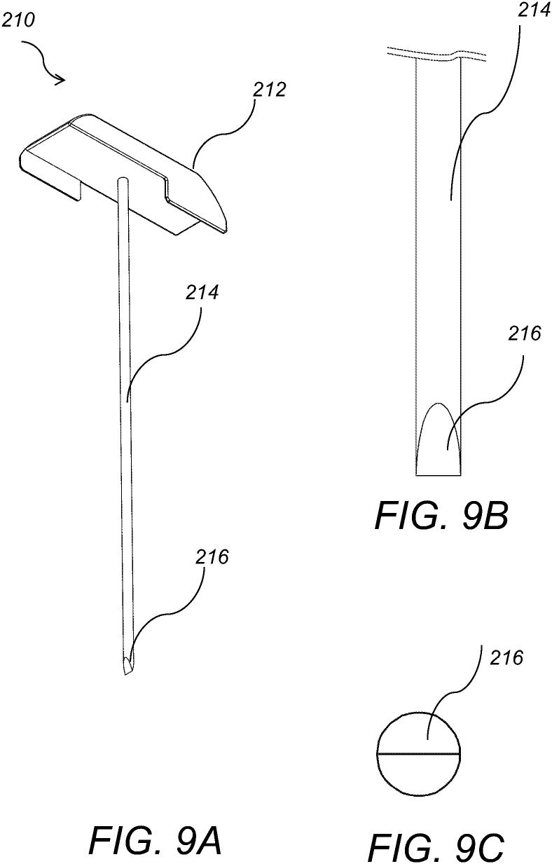

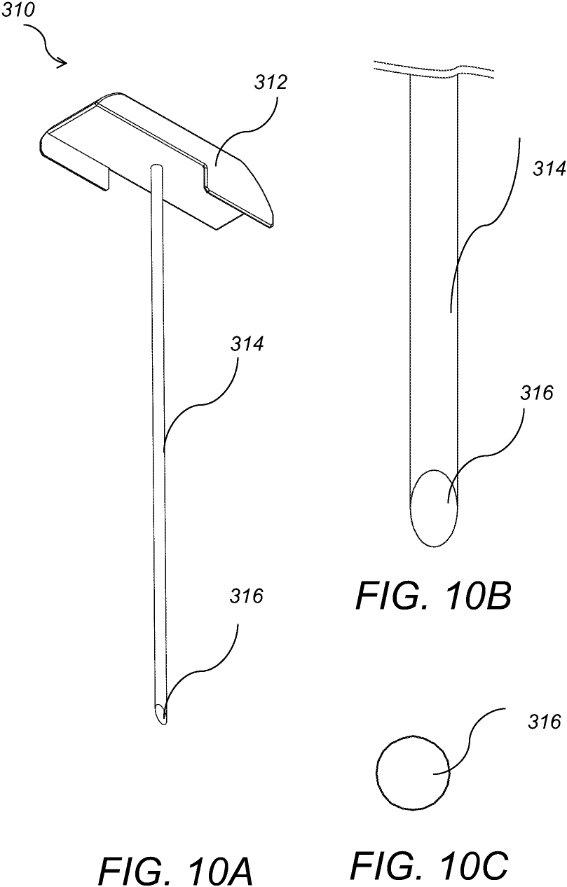

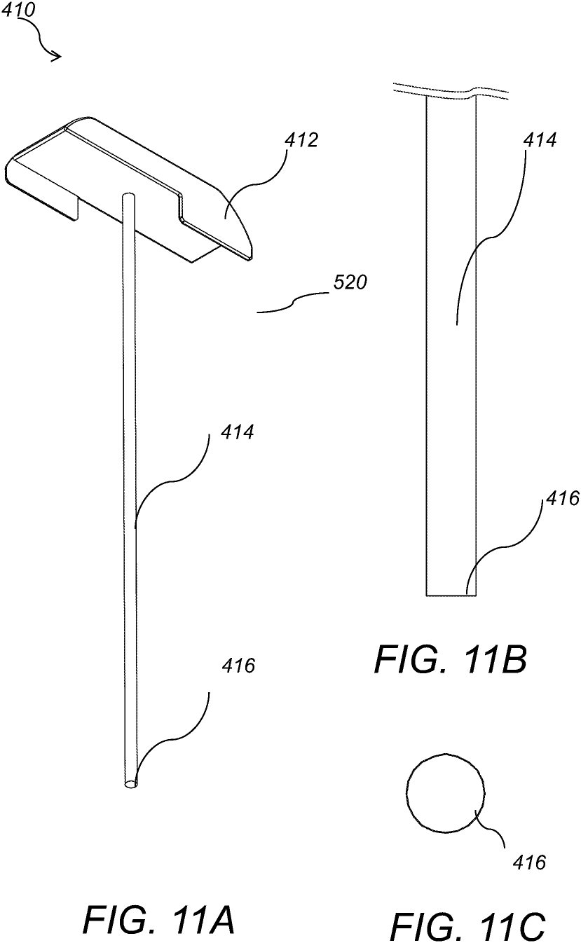

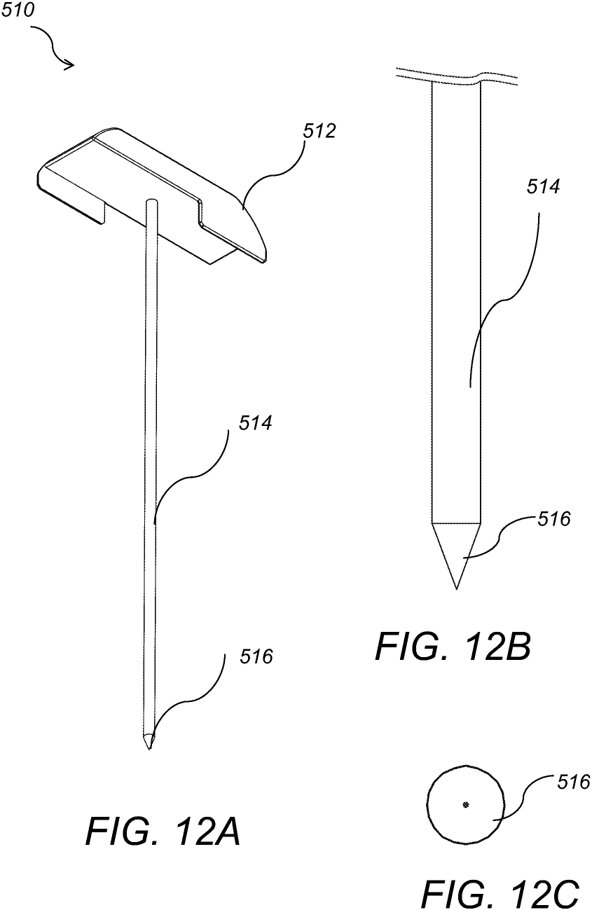

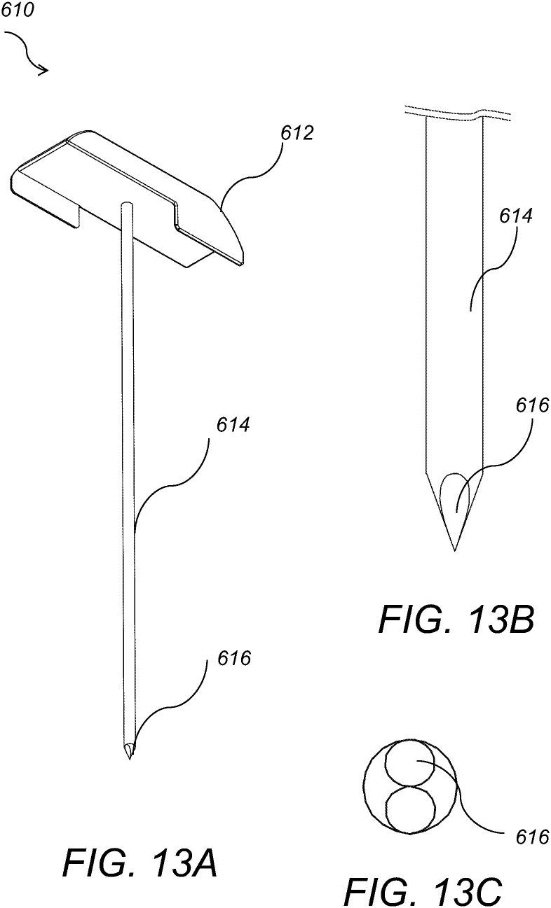

In other embodiments, the trocar needle tip 216 has a two-sided cutting blade form, as shown in FIG. 9A-FIG. 9C. In other embodiments, the trocar needle tip 316 has a bevel cutting blade form, as shown in FIG. 10A-FIG. 10C. In other embodiments, the trocar needle tip 416 has a flat form, as shown in FIG. 11A-FIG. 11C, and is used for pushing the graft material in the facet opening. In other embodiments, the trocar needle tip 516 has a cone form, as shown in FIG. 12A-FIG. 12C. In yet other embodiments, the trocar needle tip 616 has a dual round cutting form, as shown in FIG. 13A-FIG. 13C.

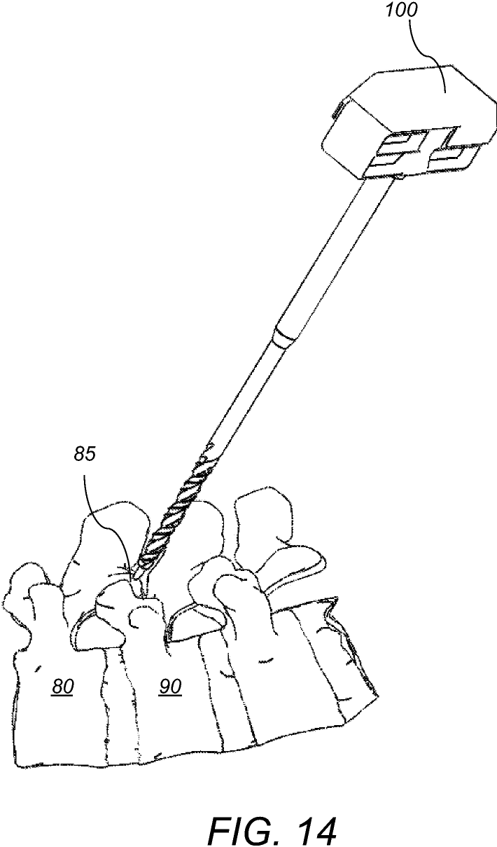

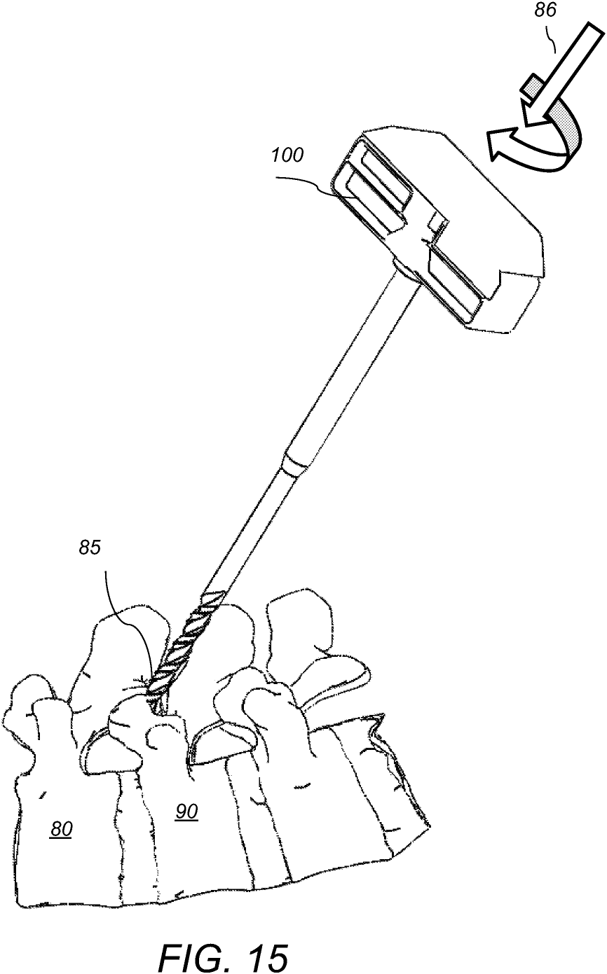

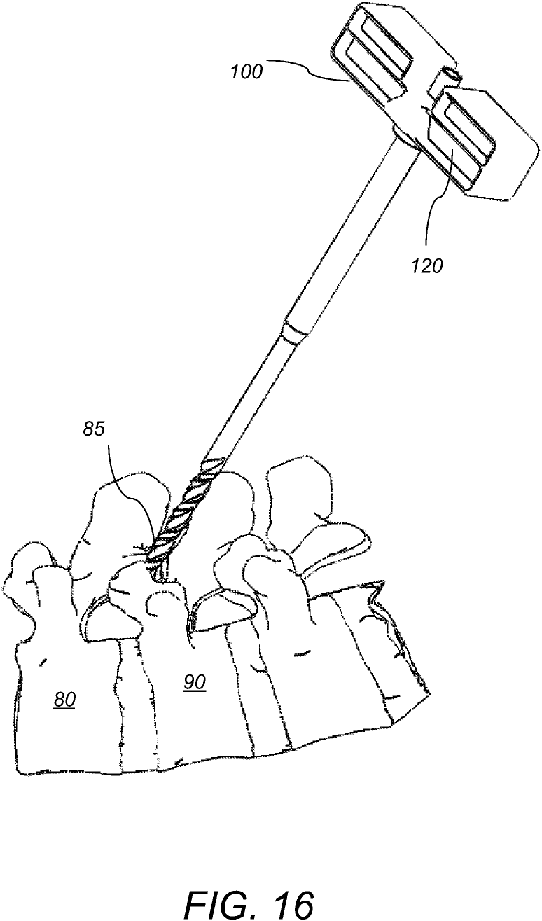

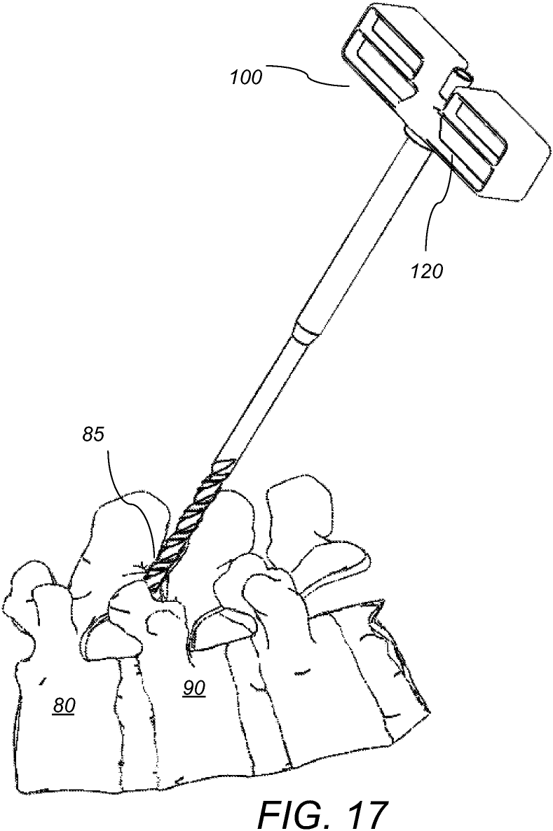

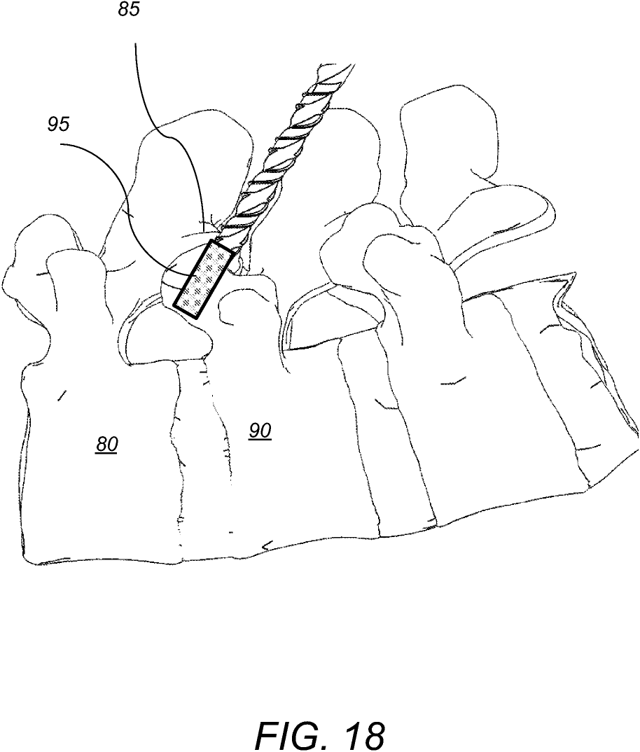

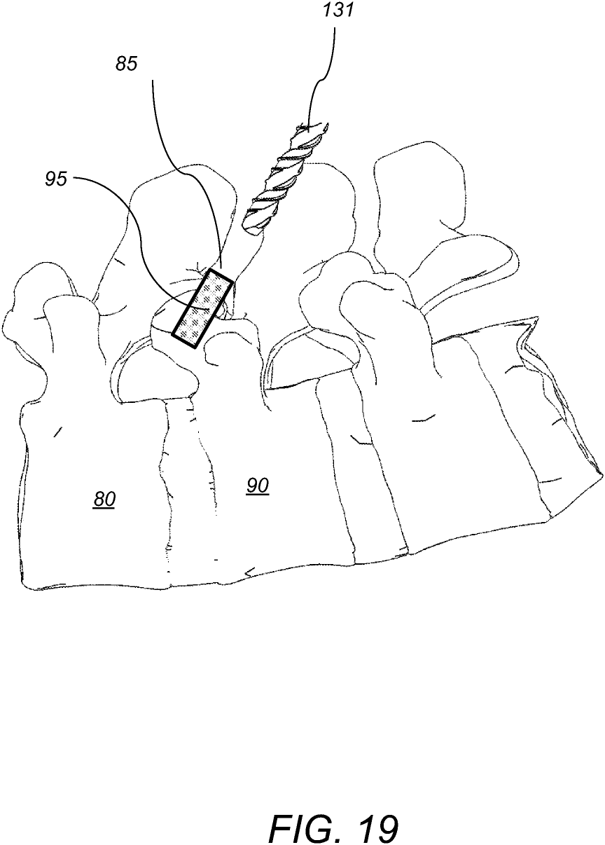

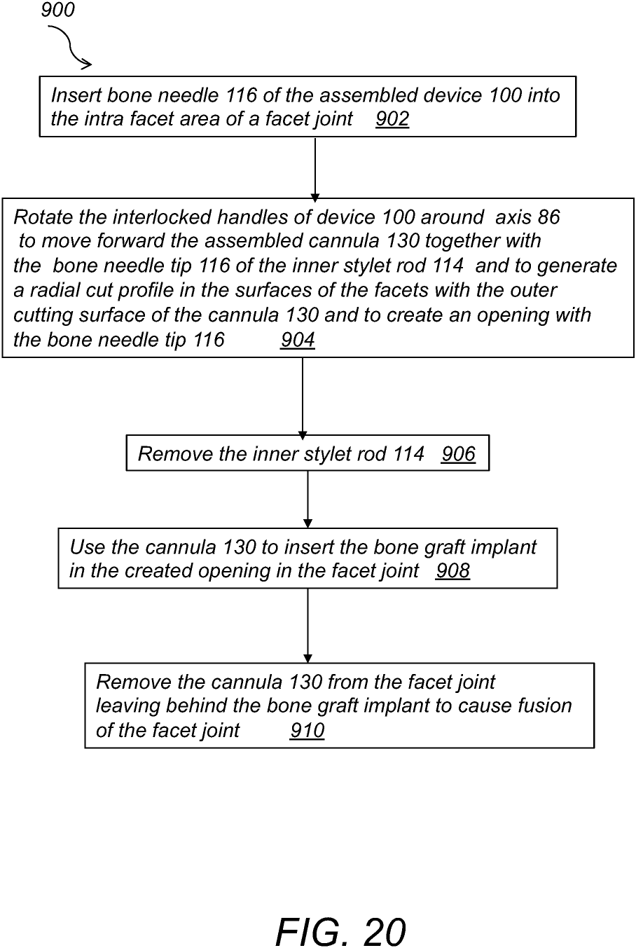

Referring to FIG. 14-FIG. 20, a method 900 for spinal facet joint fusion includes the following steps. First, inserting the bone needle 116 of the facet fusion device 100 of FIG. 1A into the intra-facet area of a facet joint 85 of two adjacent vertebras 80 and 90 percutaneously in a lateral to medial trajectory, as shown in FIG. 14 (902). Next, the two interlocked handles 112, 122 of device 100 are rotated around axis 86 in order to move forward the assembled cannula 130 together with the trocar needle tip 116 of the inner stylet rod 114 and to distract the facets, as shown in FIG. 15. The rotation of the interlocked handles 112, 122 causes the outer cutting surface 131a of the cannula 130 to cut and create the thread profile in the surfaces of the facet joint 85 and moves the trocar needle tip 116 of the inner stylet rod 114 forward to distract the facets and create an opening (904). After the opening and the thread profile are created in the facet joints, the inner stylet rod 114 is removed, and the remnants of the degenerated cartilage are removed through the cannula 130, as shown in FIG. 16 ( 906). Next, the bone graft material 95 is inserted through the cannula into the opening, as shown in FIG. 17 and FIG. 18 (908). Finally, the cannula 130 is removed while the bone graft material 95 is left in the facet joints to fuse the facet joints, as shown in FIG. 19 (910). The process is repeated for the other facet joint. This method is applied using any of the above described fixation devices 100, 200, 300, 400, 500, 600, 700, and 800 and trocar needle tips 116, 216, 316, 416, 516, and 616.

Other embodiments include one or more of the following. The allograft bone material is substituted with other biocompatible materials including synthetic bone growth promoting material, bone-polymer composite material, autograft bone material, xenograft bone material, polymers, “bio-glass” material, resorbable material, or non-resorbable material, or combinations thereof. The metallic components may be made of titanium, cobalt, stainless steel, chrome, or alloys thereof or shape- memory alloy, or ceramic-metallic composite materials, among others.

Among the advantages of this invention are the following. The devices and method of this invention provide access of a facet joint percutaneously in a lateral to medial trajectory and they are used for delivering biologics and/or synthetic materials into the facet joint through a cannulated component for the purpose of fusing the facet joint. The devices and method of this invention allow for the distraction of the facet joints, removal of the diseased cartilage, creation of channels to place bone graft and change of the formed channel trajectories while moving the device handle only radially without having to remove the cannula. The above mentioned functions are performed using only one instrument for entering the facet joint, drilling and tapping of the facet surfaces. The drilling and tapping of the facet surfaces is accomplished by using the outer surface of the cannula, without the need to insert additional instruments and by only rotating the outer handle.

Several embodiments of the present invention have been described. Nevertheless, it will be understood that various modifications may be made without departing from the spirit and scope of the invention. Accordingly, other embodiments are within the scope of the following claims.

1. A surgical device comprising:
an inner stylet component comprising a first handle and an elongated rod having a proximal end attached to a bottom surface of the first handle and a distal end comprising a needle tip;
an outer stylet component comprising a second handle and an elongated cannula having a proximal end attached to a bottom surface of the second handle and a distal open end comprising cutting elements formed on an outer surface;
wherein the elongated rod of the inner stylet component is sized to removably fit and slide within the elongated cannula of the outer stylet component;
wherein the first handle is configured to sit onto and interlock with the second handle;
wherein the elongated rod is sized so that the needle tip protrudes through the distal open end of the elongated cannula, when the first and second handles are interlocked; and
wherein rotating the interlocked first and second handles moves forward the elongated cannula and the needle tip and cuts a profile in an insertion site with the cutting elements of the outer cannula and generates an opening in the insertion site with the needle tip.

2. The device of claim 1, wherein the needle tip comprises a trocar needle tip.

3. The device of claim 2, wherein the trocar needle tip comprises a three-sided cutting conical form.

4. The device of claim 2, wherein the trocar needle tip comprises one of a two-sided cutting blade, a bevel cutting blade, a flat form, a cone form, or a dual round cutting form.

5. The device of claim 1, wherein the cutting elements of the elongated cannula are configured to cut radially and/or straight into the insertion site.

6. The device of claim 1, wherein the cutting elements of the elongated cannula comprise quad-lead cutting threads and dual-lead cutting threads.

7. The device of claim 1, wherein the cutting elements of the elongated cannula comprise a ten-lead helix cutting reamer and a chamfer.

8. The device of claim 1, wherein the cutting elements of the elongated cannula comprise a ten-lead helix cutting burr and a chamfer.

9. The device of claim 1, wherein the cutting elements of the elongated cannula comprise a quad-lead helix cutting thread that has four sharp cutting edges.

10. The device of claim 1, wherein the cutting elements of the elongated cannula comprise four straight cutting flutes and a quad-lead helix cutting thread that has four sharp cutting edges.

11. The device of claim 1, wherein the distal open end of the elongated cannula further comprises one or more fenestrations configured to be used for delivering of graft material into the insertion site.

12. The device of claim 1, wherein the inner stylet component and the outer stylet component comprise one of titanium, cobalt, stainless steel, chrome, or alloys thereof, shape- memory alloy, or ceramic-metallic composite materials.

13. A method for spinal facet joint fusion, comprising:
providing a surgical device comprising:
an inner stylet component comprising a first handle and an elongated rod having a proximal end attached to a bottom surface of the first handle and a distal end comprising a needle tip;
an outer stylet component comprising a second handle and an elongated cannula having a proximal end attached to a bottom surface of the second handle and a distal open end comprising cutting elements formed on an outer surface;
wherein the elongated rod of the inner stylet component is sized to removably fit and slide within the elongated cannula of the outer stylet component;
wherein the first handle is configured to sit onto and interlock with the second handle;
wherein the elongated rod is sized so that the needle tip protrudes through the distal open end of the elongated cannula, when the first and second handles are interlocked;
inserting the needle tip of the elongated rod into an intra-facet area of a facet joint;
rotating the interlocked handles of the surgical device to distract the facet joint, and to generate an opening with the needle tip of the elongated rod, and to cut a radial profile with the cutting elements of the elongated cannula on the intra-facet area surfaces;
removing the elongated rod and inserting graft material into the opening via the elongated cannula;
removing the elongated cannula from the facet joint leaving behind the graft material to cause fusion of the facet joint.

14. The method of claim 13, further comprising removing remnants of degenerated cartilage through the elongated cannula prior to inserting the graft material.

15. The method of claim 13, wherein the needle tip comprises a trocar needle tip.

16. The method of claim 15, wherein the trocar needle tip comprises a three-sided cutting conical form.

17. The method of claim 13, wherein the needle tip of the elongated rod is inserted into the intra-facet area of the facet joint percutaneously in a lateral to medial trajectory.

18. The method of claim 13, wherein the graft material comprises one of allograft bone material, synthetic bone growth promoting material, bone-polymer composite material, autograft bone material, xenograft bone material, polymers, “bio-glass” material, resorbable material, or non-resorbable material, or combinations thereof.

19. The method of claim 13, wherein the cutting elements of the elongated cannula are configured to cut radially and/or straight into the insertion site.

20. The method of claim 13, wherein the distal open end of the elongated cannula further comprises one or more fenestrations configured to be used for delivering the graft material into the insertion site.
